(19) United States
(12) Patent Application Publication
(10) Pub. No.: US 2010/0196483 A1
(43) Pub. Date: Aug. 5, 2010

(54) METHOD FOR TREATMENTOF SEVERE AND UNCONTROLLABLE ASTHMA

(75) Inventors: BERNHARD MUELLINGER, Munchen (DE); GERHARD SCHEUCH, Wohrathal (DE); THOMAS HOFMANN, DOYLESTOWN, PA (US); PHILIPP KRONEBERG, Olching (DE)

Correspondence Address: PETERS VERNY , L.L.P., 425 SHERMAN AVENUE, SUITE 230, PALO ALTO, CA, 94306, US

(73) Assignee: ACTIVAERO GMBH RESEARCH & DEVELOPMENT, GAUTING (DE)

(21) Appl. No.: 12/365,754

(22) Filed: Feb. 4, 2009

(51) Int. Cl. A61K 9/12; A61K 9/14

(52) U.S. Cl. 424/489; 424/45

(57) ABSTRACT

A method for treatment of severe and uncontrollable asthma by providing means for delivery of high doses of a suitable inhalable corticosteroid directly to the small airways of the lower lungs with substantial decrease in need for simultaneous administration of oral corticosteroids and with a significant reduction in undesirable secondary side effects in an oropharyngeal area. The method utilizes devices allowing individualization of treatment parameters in asthmatic patients having compromised breathing pattern due to severe and uncontrollable asthma.

### BACKGROUND OF THE INVENTION

1. Field of the Invention

This invention concerns a new and improved method for treatment of severe and uncontrollable asthma by providing means for delivery of high doses of a suitable inhalable corticosteroid directly to the small and central airways of the lower lungs without need for simultaneous administration of oral corticosteroids or with a significantly decreased need for such simultaneous administration of oral corticosteroids. The method significantly increases delivery of the aerosolized inhalable corticosteroid into the bronchi, bronchioli, and alveoli of the central and lower peripheral lungs and decreases deposition of the corticosteroid into the bronchi and trachea of the upper lungs as well as in an oropharyngeal area and thereby significantly decreases or completely eliminates undesirable secondary (e.g. oropharyngeal) symptoms associated with delivery of high doses of inhalable corticosteroids. The method utilizes devices allowing individualization of treatment parameters in asthmatic patients having compromised breathing pattern due to severe and uncontrollable asthma.

2. Background and Related Disclosures

Asthma is a major cause of chronic morbidity and mortality throughout the world and is one of the most chronic diseases, with estimated 300 million individuals affected by this condition.

People suffering from asthma may have either a mild form of asthma that is easily controlled with oral, systemic or inhalation therapy or a severe form of asthma that is difficult to control and treat. The severe form of asthma is connected with a heightened bronchial hyper-reactivity and with chronic severe and uncontrollable asthmatic symptoms.

Many attempts have been made to control asthma with a particular emphasis on a control and treatment of patients suffering from the severe and uncontrollable asthmatic attacks. However, since each individual is unique in his/her degree of reactivity to environmental triggers, asthma affects each patient differently. This naturally influences the type, dose and a route of administration of various medication and treatments.

Global Initiative for Asthma (GINA) asthma guidelines have been established to determine severity of the asthma. Severe and uncontrollable asthma is classified by GINA guidelines as steps IV and step V, generally requiring administration of oral corticosteroids in combination with inhaled corticosteroids. For step IV, the preferred treatment is to combine medium to high doses of inhaled corticosteroid with a long-acting inhaled β-agonists. For step V, the above medication is further supplemented with orally administered glucocorticosteroids. Both treatments are known to cause or be associated with severe side effects, and these side effects may be exacerbated with a prolonged use of high-doses of inhaled corticosteroids.

As indicated already above, many attempts to successfully treat severe and uncontrollable asthma have been made. These attempts include development of new and more potent drugs, such as for example more potent corticosteroid fluticasone as well as new nebulizing technologies that affect pulmonary drug delivery.

Eur. J. Clin. Pharmacol, 57:637-41 (2001) describes a study comparing a large volume spacer and fluticasone nebulizer (FP-neb) in delivery of fluticasone propionate by inhalation in healthy volunteers. The large volume (750 ml) spacer was shown to produce about a sevenfold higher relative lung dose than nebulizer. This reference shows that the efficacy of the aerosol delivery depends on the device used for such delivery.

Respir. Med., 93(10):689-99 (1999) describes an oral steroid-sparing effect of high dose (4000 μg/day/bid) of inhaled fluticasone propionate. Reduction in orally administered prednisone was significantly greater in the group receiving 4000 μg of fluticasone propionate per day than 1000 μg per day. However, it is noticeable that using this technology, high percentage (37%) of all patients discontinued 4000 μg treatment, presumably for high occurrence of severe side effects.

J. Allergy Clin. Immunol., 103:267-75 (1999) describes an oral corticosteroids-sparing effect and improved lung function in patients with severe chronic asthma who received 500 or 1000 μg of fluticasone propionate administered twice daily. While this treatment eliminated a need for oral prednisone, topical adverse effects associated with inhaled corticosteroids were observed during this treatment.

Br. J. Clin. Pract., 48:15-8 (1994) assessed a long-term safety of fluticasone propionate in asthmatic children. Adverse effects were reported by 51% of patients even with such low doses as 50 or 100 μg administered twice a day via a dry powder inhaler.

Cochrane Database Syst. Rev., 2:CD002310 (2004) reviewed a potency of fluticasone propionate for treatment of chronic asthma and compared its effect to that of beclomethasone and budenoside. The study showed that fluticasone propionate, given at half the daily dose of beclomethasone or budenoside, resulted in improvement of forced expiratory volume in the first second (FEV1). Unfortunately, due to a larger deposition of the fluticasone in the upper lungs, it also had a higher risk of pharyngitis and other adverse side effects.

Cochrane Database Syst. Rev., 3:CD003534 (2005) describes use of inhaled fluticasone at different doses. While patients receiving 2000 μg per day of fluticasone propionate were more likely to reduce a need for oral prednisolone then those on 1500 or 1000 μg/day, hoarseness and oral candidiasis were significantly greater for these higher doses.

Respiratory Medicine, 94: 1206-1214 (2000) investigated the efficacy and safety of nebulized fluticasone propionate compared to orally administered prednisolone. The nebulized fluticasone was at least as effective as oral prednisolone in the treatment of children with acute exacerbated asthma.

Cochrane Database Syst. Rev., 4:CD004109.pub2 (2008) evaluated the efficacy of an initial high dose of inhaled corticosteroids compared to a lower to moderate dose. Authors concluded that treatment should commence with a moderate rather than high dose of inhaled corticosteroids.

Annals Allergy, Asthma and Immunology, 92:512-522 (2004) reviewed the efficacy and safety of inhaled corticosteroids when used to reduce daily oral corticosteroid requirement in patients with severe asthma. Authors concluded that inhalable corticosteroid can reduce orally administered corticosteroids requirements in patients with persistent and exacerbated asthma. However, the question of increased adverse side effects still remains.

Respiratory Medicine, 93: 689-699 (1999) investigated the steroid-sparing effect of two doses of nebulized fluticasone propionate in patients with severe chronic asthma. The nebulized fluticasone at a daily dose between 1 and 4 mg was safe and effective means for reducing the oral steroids requirement of patients with chronic oral dependent asthma.

Disclosures discussed above indicate that a need for orally administered steroids in patients suffering from severe and uncontrollable asthma may be decreased by administration of appropriately high doses of inhalable corticosteroids. However, when such high doses of inhalable corticosteroid are administered, severe adverse side effects occur, preventing a truly efficacious treatment of these patients.

It would, therefore, be advantageous to have available a method that would provide efficacious treatment for severe and uncontrollable forms of asthma with a high dose of the inhalable corticosteroid deposited at a site of asthmatic inflammation, namely in alveoli and bronchiole of the lower lungs combined with a low deposition of the corticosteroid in the trachea and in oropharyngeal area, wherein orally administered steroids could be eliminated or, at least, an oral dose of these drugs could be significantly reduced and wherein said treatment would be able to selectively deliver higher doses of corticosteroids into lower peripheral lung of an asthmatic patient without depositing said drug into a mouth or pharyngeal cavity.

It is, therefore, a primary object of this invention to provide a method for efficacious treatment for severe and uncontrollable forms of asthma by providing means for delivery of a sufficiently high dosage of a corticosteroid for treatment of said severe uncontrolled asthma and wherein an oral delivery of corticosteroids could be either completely eliminated or reduced by at least thirty percent, in mean, and wherein said treatment would be able to deliver higher dosages of corticosteroid selectively into alveoli and bronchi of the lower peripheral lungs of an asthmatic patient without depositing said drug into a mouth or pharyngeal cavity or causing other undesirable adverse side effects.

### SUMMARY

One aspect of the current invention is a method for treatment of severe uncontrollable asthma by providing means for increasing efficacy of an inhalable corticosteroid delivery by delivering large dosages of such inhalable corticosteroid selectively to alveoli and bronchiole of a lower lungs of a patient suffering from severe uncontrollable asthma without causing adverse side effects and secondary symptoms by incidental delivery of these corticosteroid to a mouth cavity, throat and upper lungs.

Another aspect of the current invention is a method for treatment of severe uncontrollable asthma by providing means for delivering a larger percentage of dosages of a corticosteroid selectively to the lower lungs of a patient suffering from severe uncontrollable asthma and thereby eliminating or significantly reducing a need for concurrently administered corticosteroids orally or systemically.

Still another aspect of the current invention is a method for treatment of severe uncontrollable asthma with completely eliminated or with at least 30% reduced dose of orally delivered corticosteroid by providing means for treating such severe and uncontrollable asthma with a high dosage of a corticosteroid delivered as an aerosol having particle sizes predominantly from about 2 to about 6 μm, by nebulization using a nebulizing system that applies, during delivery into a patient's lungs, an overpressure enabling such delivery in less than 6-10 minutes and further resulting in selective delivery and homogeneous distribution of the corticosteroid in the lower lungs, wherein said nebulizing system is equipped to have a controlled airflow, and defined volume.

Yet another aspect of the current invention is a method for treatment of severe uncontrolled asthma with nebulized fluticasone, budenoside, beclomethasone dipropionate, budenoside, mometasone furoate, ciclesonide, flunisolide or triamcinolone acetonide wherein the clinical effect is reached without increasing systemic and local extrathoracic or oropharyngeal side effects.

Still yet another aspect of the current invention is a method for treatment of severe uncontrolled asthma by inhalation of nebulized fluticasone as a representative corticosteroid administered into lungs via nebulizer with concentration of fluticasone in the nebulizer being higher than 200 μg/ml, preferably 0.5-2 mg/mL, formulated as a suspension and with total filling dose of fluticasone not exceeding about 4000 μg, using a nebulizing system that applies overpressure during inhalation and thus assures selective deposition of more than 200 μg of said corticosteroid into the lower lungs in less than 6-10 minutes.

Still yet another aspect of the current invention is a nebulizing system comprising a device able to provide an overpressure as well as a controlled air flow during a patient's inspiration time to reduce patient breathing effort with pressure at the nebulizer mouthpiece up to a positive pressure of between 0-40 mbar.

Another aspect of the current invention is a method for treatment of severe uncontrollable asthma by providing a nebulization protocol wherein, during one inspiration time and under the mean inspiratory flow rate equal to or below 20 l /min, the patient is subjected to a first volume of 150 ml or less of particle free air in a predetermined time of less than 0.5 sec, followed by a second volume of between about 200 and about 3000 ml of an aerosol containing an inhalable corticosteroid administered in a predetermined time from about 1 to about 10 seconds, said aerosol preferably administered within less than 0.2 sec after the start of inspiration, followed by a third volume of between about 100 to about 500 ml of particle free air, and wherein such protocol results in forcing said inhalable corticosteroid from the extrathoracic and tracheal airways to be deposited more selectively into the lower airways.

Yet another aspect of the current invention is a method for treatment of severe uncontrolled asthma by inhalation of nebulized corticosteroid in an aerosol having particle sizes predominantly in the range from about 2 to about 6 μm, preferably from about 3 to about 5 μm.

Another aspect of the current invention is a method for treatment of severe uncontrollable asthma by providing means for delivering larger dosages of a corticosteroid once a day selectively to the lower lungs of a patient thereby eliminate or significantly reduce a need for concurrently administered corticosteroid orally or systemically wherein asthma is improved without loss of FEV1 and with diminished adverse side effects.

Still another aspect of the current invention is a method for treatment of severe uncontrollable asthma by providing a nebulization system for individualization of the treatment wherein said nebulization system comprises a preprogrammable volume for drug delivery, a preprogrammable air flow delivery, a preprogrammable overpressure, and may further comprise a compliance monitoring system which allows the patient and the doctor to see and control frequency of the corticosteroid delivery, such means being any storage media, a smart card, a chip or a wireless communication connection that permits evaluation of the treatment during and after the end of a treatment period and determination of frequency of the corticosteroid administration.

### DEFINITIONS

As used herein:

“Inhalable corticosteroid” means a corticosteroid that is suitable for delivery by inhalation. Exemplary inhalable corticosteroids are fluticasone, beclomethasone dipropionate, budenoside, mometasone furoate, ciclesonide, flunisolide, triamcinolone acetonide and any other corticosteroid currently available or becoming available in the future.

“Oral steroid” means any steroid that is suitable for oral or systemic treatment of asthma. Representative steroid are prednisone, prednisolone, methylprednisone, dexamethasone or hydrocortisone.

“lower lungs”, “small lungs” or “peripheral lungs” means an area of the lungs primarily containing bronchi, alveoli and bronchiole, a primary site of asthmatic inflammation, narrowing and constriction. Large and selective depositions of an inhalable corticosteroid in this area is eminently desirable and contributes to an efficacious treatment of severe uncontrolled asthma.

“Upper lungs”, “central lungs” or “large lungs” means an upper area of lungs containing bronchi and trachea. Large depositions of an inhalable corticosteroid in this are undesirable as they are leading to development of adverse side effects.

“Oropharyngeal area” or “extrathoracic area” means a mouth, nose, throat, pharynx and larynx. Any deposition of the inhalable corticosteroid in these areas is undesirable and leads to development of severe adverse effect such as hoarseness, loss of voice, laryngitis and candidiasis. It is preferable that there is none or a very small residual deposition, occurring primarily during expiration of the inhaled corticosteroid, in this region.

“One breath” means a period of time when a person inhales (inspires) and exhales during a regular breathing pattern that includes inhaling and exhaling.

“Inspiration time” or “inspiration phase” means a fraction of one breath when a person inhales an air or, in this instance, an aerosolized corticosteroid. For purposes of this invention, the aerosolized corticosteroid is administered to an asthmatic patient during the inspiration time either with a slight overpressure to force the aerosol to the lower lungs without a large deposition of the drug in the upper lungs and oropharyngeal area using the AKITA protocol and nebulizer, or as a second volume between the first and second volume of delivered air without particles using a breath actuated nebulizer and protocol.

“Expiration time” means a fraction of one breath when a person exhales the air, nitric oxide or another metabolite from the lungs. For the purposes of this invention, it is preferable that the aerosolized drug is forced with a slight overpressure into the lower lungs during inspiration and that it is not exhaled during expiration time or that only a small portion is exhaled.

“Bolus technique” means transportation of the corticosteroid aerosol to a predefined region in the lungs.

“FEV1” means forced expiratory volume in one second.

“VC” means vital capacity.

“ERV” means expiratory resting volume.

“Particle-free air” means the air that does not contain any drug and is delivered before and after the aerosolized drug delivery.

“Overpressure inhalation” means inhalation with actively provided air that is preferably predefined in airflow for a predefined time. During inspiration the patient adjusts to the inspiratory flow rate. If the patient inhales more passively an overpressure of up to 40 mbar is applied during the inhalation phase to reduce the inspiratory effort. Consequently, the patient is able to inspire a more deep inhalation volume and inhale with a slower inspiration flow rate compared to a spontaneous inhalation.

### DETAILED DESCRIPTION OF THE INVENTION

The current invention relates to a method for treatment of severe and uncontrollable asthma by providing a means for delivery of high doses of a suitable inhalable corticosteroid directly to the small airways of the lower lungs without need for simultaneous administration of oral corticosteroids or with decreased need for such simultaneous administration of oral corticosteroids. The method significantly increases delivery of the aerosolized corticosteroid into the alveoli and bronchioles of the lower peripheral lungs and decreases deposition of the corticosteroid into the bronchi and trachea of the upper lungs as well as in an oropharyngeal area and thereby significantly decreases or completely eliminates undesirable secondary symptoms. The method utilizes devices allowing individualization of a delivered volumetric flow and vaporized aerosol together with a controlled airflow and with airflow overpressure conditions in asthmatic patients with compromised breathing pattern.

I. Severe and Uncontrollable Asthma

Asthma is a chronic inflammation of the bronchial tubes of airways that causes swelling, bronchial narrowing and constriction. As a consequence, patients suffering from asthma have difficulty breathing. The bronchial swelling, narrowing and constriction is generally treated with oral or inhalable drugs, preferably with inhalable steroids, such as fluticasone, budenoside, beclomethasone, mometasone, ciclesonide, flunisolide, triamcinolone acetonide and any other corticosteroid suitable for inhalation therapy.

A mild form of asthma may be easily controlled and treated with a great variety of oral, systemic or inhalation therapies. Severe forms of asthma are characterized with a heightened bronchial hyper-reactivity and with other chronic symptoms. Treatments for the individuals suffering from the severe uncontrollable asthma are very difficult and complex.

A. Currently Available Treatments

The currently available treatments for asthma are largely dependent on the severity of the disease. In most cases, these treatments involve administration of steroids, whether orally administered corticosteroids (OCS), such as prednisone or prednisolone, or inhalable corticosteroids (ICS), such as fluticasone, beclomethasone, budenoside, mometasone, ciclesonide, flunisolide or triamcinolone acetonide in a therapeutic dose. These treatments may be, in some case, supplemented with other drugs, such as, for example, bronchodilators as β-agonists. Since the orally or otherwise systemically delivered corticosteroids cause rather severe adversary side effects and secondary symptoms in the patients and their systemic delivery affects the whole body, the locally administered corticosteroid by inhalation are highly preferred as a current treatment for asthma.

The moderate and more severe asthma patients, including the pediatric and geriatric asthma population, are frequently treated with nebulized inhalable corticosteroids using jet or ultrasonic nebulizers. These nebulizers typically deliver a filling volume of 1 or 2 ml of liquid suspensions containing about 200 micrograms of inhalable corticosteroids and maximum up to 2000 micrograms. Inhalable corticosteroids are also delivered by metered dose inhalers (MDI) and dry powder inhalers (DPI), at nominal doses at around 100 micrograms. These doses are mostly sufficient for treatment of mild forms of asthma where the quantity of the delivered dose is not critical for ameliorating asthmatic symptoms.

For severe asthmatic forms, however, the quantity of the dose delivered to the site of the asthmatic inflammation is often critical and decisive of the successful treatment. The currently recommended nominal doses for efficacious treatment of severe asthma range between 400 and 1600 μg. The amount to be deposited at a site of inflammation, in alveoli and bronchioles of the lower lungs, are in the order of 10-25% of the above nominal dose resulting in a maximum deposited lung dose of 250 micrograms. It would be an advantage to deliver and deposit between 400 and 800 micrograms in the lungs and mostly in the lung periphery.

Unfortunately, due to their inefficiency, none of the currently available nebulizing system is able to deliver such a dose into the lower lungs without causing serious adverse reactions. The currently available nebulizers typically deliver only about 5% and up to maximum of 10% of the total dose of the corticosteroid placed in the nebulizer. Since conventionally corticosteroid suspensions are difficult to nebulize, most of the drug remains in the nebulizer. Therefore, the effectiveness of nebulizers for corticosteroid delivery is much lower compared to their effectiveness for inhalation solutions. Additionally, many other disadvantages are observed with currently available treatments, particularly as those treatments concern a treatment of severe and uncontrollable asthma.

Disadvantages of the Currently Available Treatments

Primary disadvantages of the currently available treatments with steroids are connected with pharmacological effects of steroids, particularly when delivered orally or systemically and not directly to a targeted organ needing such treatment. Such oral or other systemic administration of steroids affects the whole body with targeted organ receiving only small amounts of the administered drug. This, of course, results in a need for administration of large doses of the steroids. Because of the overall pharmacological effect of orally and systemically delivered steroids, a targeted topical administration would seem to be a more preferred route of steroid administration. However, such targeted topical delivery of steroids by inhalation is also not without problems.

First, the currently used nebulizers typically deliver only a fraction of a total dose placed into the nebulizer. Thus, for example, from a total dose of 2000 micrograms placed into the nebulizer before aerosolization, only about 5-10% of the total dose is actually deposited at a site of the asthmatic inflammation in the alveoli and bronchiole of the lower lungs and therefore the actual deposited dose at such site is only about 100 and at maximum 200 micrograms. This dose is insufficient to treat severe asthma. The remaining 90-95%, that is 1800-1900 micrograms, of the drug is either deposited in the upper lungs, or most of it is deposited in the oropharyngeal area (causing oropharyngeal side effects, such as candida infection or hoarseness), or it is exhaled or it remains in the nebulizer and is wasted.

Second, a dose delivered to a site of asthma is too low to achieve an efficacious treatment of asthma, particularly in patients with severe and uncontrollable asthma. When the dose filled in the nebulizer is increased to above 200 and preferably between 400 and 2000 micrograms, given twice daily, the actual deposited dose in the lower lungs is still only 5-10% that is only 20 to a maximum of 200 micrograms. Such amount is not only insufficient to treat severe and uncontrolled asthma, but more importantly such delivery is accompanied by hoarseness, alteration of voice, laryngitis, candidiasis and irritation of the upper lungs and oropharyngeal area due to a large portion of the nebulized dose of the steroid having been incidentally deposited there.

Third, the inhaled steroid dose filled into the device cannot be increased above 2000 micrograms when using conventional nebulizers, because of the severe side effects. The undesirable side effects, such as candidiasis, soreness, hoarseness, laryngitis or voice alteration are observed even with the low doses of inhalable corticosteroids. The reason for this is the high mouth and throat deposition of the corticosteroid with the currently used inhalation systems. Whereas only a small fraction of the inhaled drug dose reaches the lungs, a large amount of the dose will be lost or deposited in the oropharynx and upper lungs. This is the major reason why the inhaled dose in these patients cannot be further increased.

Fourth disadvantage of the currently available treatment is a patients' compliance with a nebulizer use. The inhalation devices which are currently used and available will deposit a maximum of 200 micrograms in the lungs only when the patients are inhaling appropriately. It is well known that only a few of the patients using an inhalation device are using it correctly. Commonly observed mistakes by the patients during an inhalation maneuver are: breathing is too fast, breathing is too shallow or the breathing is not coordinated. When the patient breathes too fast, inhalation flow rate results in extremely high drug deposition in the back of the throat and the larynx, with almost no drug deposition in the deep lungs. When the patient's breathing is too shallow, a shallow breath takes on only a small inhalation volume that cannot transport the aerosolized drug particles deep into the lungs thereby resulting in minimal deep lung deposition. When the breathing is not well coordinated, for example when the aerosol production and the inspiration phase of a patient are not in line, there is no or lesser lung deposition. Moreover, some patients only take the drug when asthmatic symptoms occur and not continuously in a controllable manner. Patients with a poor breathing maneuver seldom profit from the inhalation therapy, because they generally do not get a sufficient amount of the drug into the lungs. Since these patients also have more side effects caused by the high extrathoracic deposition, an abortion of and withdrawal from inhaled corticosteroid therapy is more likely.

Fifth, because currently there is not available a reliable method for treatment of patients suffering from severe and uncontrollable asthma, these patient are often condemned to taking oral steroids that, of course, assert their strong effect over the whole body of the patient and are not limited to lungs where the underlying disease is.

II. Method For Treatment of Severe and Uncontrollable Asthma

The method for treatment of asthma, particularly severe and uncontrollable asthma according to the current invention provides several advantages over the currently available treatments.

The method allows a deposit of high doses of inhalable corticosteroids in the lower lungs of patients, without increasing corticosteroid deposition in the mouth, throat and lower lungs and provides for the drug aerosolized particles to be deposited deep into the lungs during patients breathing. The method provides an aerosol having the optimal particle sizes for homogenous deposition of the drug in the lower lungs that prevents high losses of drug in the oropharynx. During the inhalation, the nebulizer provides a slight overpressure during delivery of the aerosol to allow preferable deposition of the aerosolized drug into the deep lung and prevent exhalation of aerosol during the exhalation phase.

The method defines a partition of one breath into two fractions, namely an inspiration time and expiration time wherein during the inspiration time a so called bolus technique is used to transport the drug containing aerosol to a predefined region in the lungs and, during the expiration time, to exhale a minimum of the drug from the lungs at end of the breath. The method of the invention results in a high deposition of between 400 and 1000 micrograms of the total 2000-4000 micrograms of corticosteroid drug filled in the nebulizer into the lower lungs of the patient in less than 6 to 10 minutes, in average, and permits decreasing or eliminating need for administration of orally administered steroid concurrently with the inhalation treatment.

A. Deposited Doses

The current method enables deposition of 2-4 times higher percentage of the total drug placed in the nebulizer in the lower lungs than previously possible. When the prior available methods achieved deposition of only about 200 micrograms of the corticosteroid in the patient's lung from the total dose of 2000 micrograms supplied to the nebulizer, the current method achieves between 400 and 1000 microgram deposition, all of it preferentially in the lower lungs. Moreover, such dose is sufficient for replacing a twice daily dosing with 2000 mg of the corticosteroid with once a day dose of the 2000 micrograms, or with a twice a day dose of 1000-2000 micrograms each to achieve amelioration of severe asthmatic symptoms. Additionally, the amount of the corticosteroid in the lower lungs is significantly larger compared to the amount of corticosteroid deposited in the upper lungs and particularly in oropharyngeal area.

B. Undesirable Adverse Side Effects

The method allows deposition of the larger amount of the corticosteroid in the lungs without many undesirable adverse side effects previously observed with administration of lower amounts of the drug in the lower peripheral lungs. The previously observed side effects, such as hoarseness, soreness, loss of voice, laryngitis or candidiasis due to the deposition of large amounts of the corticosteroid in the mouth and throat are suppressed or not observed with the current method.

C. Particle Sizes of the Aerosol

The method provides an aerosol having the optimal particle sizes for homogenous deposition in the lower lungs to prevent high losses of drug in the oropharynx as well as losses in upper lungs. The method, therefore, provides for an aerosol having sizes of aerosolized particles corresponding substantially to a size of the alveoli and bronchiole. The right particle size for targeting the alveoli and bronchiole is between 2 and 5 microns. Particles larger than that are selectively deposited in the upper lungs, namely bronchi and trachea and in the mouth and throat, i.e. oropharyngeal area. Consequently, the method provides for aerosol to be limited to particle sizes between 2 and 6 microns, preferably to particle sizes between 3 and 5 microns (MMAD) with geometric standard deviation (GSD) of less than 2.5.

D. Delivery of the Aerosol Under Overpressure

The method provides means to deliver the corticosteroid aerosol under overpressure no higher than 40 mbar. Such slight overpressure allows the aerosol to be actively forced to the smaller lungs without causing damage to the lungs. Such overpressure is typically achieved with a compressor or pump unit attached to the nebulizing device where such unit is optionally further equipped with a timer so that the overpressure period is limited strictly to a fraction of the inspiration time when the corticosteroid is delivered. In another embodiment, the overpressure is initiated by a patient's inspiration time breathing. When the patient inspires with overpressure, the patient's breathing effort is reduced. Consequently, patients with severe asthma are able to perform a deeper and slower breathing pattern, compared to spontaneous inhalation without overpressure.

During the inhalation, the nebulizer provides a slight overpressure to the aerosol to allow preferable deposition of the aerosolized drug into the deep lung and prevent its removal during expiration. During expiration, the overpressure is not applied and the patient exhales normally, without any airflow or pressure being applied.

E. Bolus Technique

The method defines a partition of one breath into two fractions, namely an inspiration time and expiration time wherein during the inspiration time a bolus technique is used to transport the drug containing aerosol to a predefined region in the lungs and, during the expiration time, to expire a minimum of the drug from the upper lungs and from oropharyngeal area. In some embodiments, the inspiration time may be further divided into subfractions where the particle free air is delivered before and after the aerosol delivery of the corticosteroid.

F. Delivery Time

The method provides for shorter delivery time than conventional nebulizer for the same drug amount deposited in the lungs. Typically, the delivery of the 200 micrograms of the corticosteroid would take between 5 to 20 minutes using a conventional jet nebulizer. The current method provides for a deposition of more than 400 micrograms in the lungs in less than 10 minutes, preferably in less than 6 minutes.

G. Weaning From Oral Corticosteroids

Finally, the current method permits weaning of the asthmatic patient from the oral corticosteroids. With improved local delivery of the drug to the lower lungs in 2-4 times higher doses achieved with once daily administration, the patients are able to withdraw from oral corticosteroids treatment in from two to about five weeks, during which time the oral dose of the corticosteroids is slowly being decreased. Moreover, such withdrawal is possible without decrease in FEV1 and in some instances with actual increase of the FEV1 and without significant adverse reactions. This is particularly true when the used corticosteroid is fluticasone administered in 2000 micrograms/day dose.

Typically the degree and onset of asthma improvement is observed in 2-5 weeks of treatment. Such improvement occurs without necessity of oral corticosteroid and often results also in improved pulmonary functions such as increased FEV1 and with decreased inflammation.

III. Treatment Protocol

The actual treatment protocol for treatment of severe asthma according to the invention consists of several steps that need to be undertaken.

A. Protocol Steps

First, the patient is evaluated for severity of the asthma under Global Initiative for Asthma (GINA) guidelines. Asthmatics with severe and uncontrollable asthma are classified as step IV or step V, described as a serious chronic condition often with exacerbation. Oral corticoid dose (OCS), asthma control, exacacerbations, FEV1, exhaled nitric oxide, vital capacity and other pulmonary Functions are measured and recorded.

A mode and a regimen of the treatment is determined. Such mode of the treatment typically involves an initial combination of the inhalable and oral treatment with corticosteroids and optionally in combination with bronchodilators as β₂-agonists or other drugs, as appropriate. Typically, the patient may be treated with up to 150 mg of prednisone or prednisolone/day. The regimen for the treatment involves determination of an appropriate inhalable corticosteroid, appropriate daily dose and appropriate daily delivery once, twice or in rare circumstances, three times per day. For patient's convenience and practicality, once daily (QD) delivery is most preferable to twice a day (BID) or three times per day (TID).

Once the mode and regimen of the treatment is determined, an appropriate method for treatment is selected. Two possible methods are available under the method of the current invention. One method involves an inhalation system for control of breathing pattern (AKITA protocol) and comprises a use of the AKITA system, as described below. Another system involves use of a breath actuated nebulizer, also described below.

A patient is then treated once daily, in rare instances twice daily, with more than 200 up to 4000 micrograms of an inhalable corticosteroid, depending on the selected inhalable corticosteroid. The inhalable corticosteroid is selected from the group consisting of fluticasone, flunisolide, beclomethasone dipropionate, budenoside, mometasone furoate, ciclesonide and triamcinolone acetonide. The preferred treatment consists of a 2000 microgram of fluticasone propionate administered once/twice per day using the AKITA nebulizer or breath actuated nebulizer. Although the exact efficacious dose for treatment of severe asthma for each inhalable corticosteroid is not known, most likely, the effective deposited dose for severe asthma is >400 micrograms, and even more likely around 600-1000 micrograms. The treatment with concurrent oral corticosteroids is continued but is gradually decreased to doses lowered by at least 30% of the initial oral dose or eliminated altogether. Typically, such decrease occurs within 2-3 weeks. Complete elimination of the steroid may happen in 3-5 weeks or may take longer.

Treatment continues daily and the patient is periodically evaluated for pulmonary functions including FEV1. When the patient's pulmonary functions are stabilized with an initial oral dose of the steroid, the oral dose of the steroid is lowered and maintained until patient's pulmonary functions are again stabilized. This process continues until the patient is weaned from the oral corticosteroid completely or until the patient is stabilized at certain low level of oral corticosteroid.

Typically, the oral dose of the steroid is decreased by at least 30% of the initial dose but is preferably completely eliminated.

B. Inhalation System (AKITA) For Control of Breathing Pattern

When the AKITA protocol is selected for treatment, the patient is provided with the AKITA nebulizing system, as described below.

The selected corticosteroid in the predetermined amount is placed into the device nebulizer. For example, 2000 micrograms of fluticasone is filled in the nebulizer in form of an aqueous suspension or in form of 1 to 5 ml solution. The corticosteroid is most likely formulated as a suspension, and contains a steroid concentration around 1 mg/mL. Alternatively, the effective amount can be administered in form of dry powder, via disagglomeration of the powder, and with effective airflow control. The nebulizer is directly connected with the mouthpiece that is further equipped with pressure sensor connected with a compressor. Inhalation period (inspiration time) may be preset to a pattern comfortable for a patient, for example, from 1 to about 10, preferably about 3-4 seconds of inspiration time. When the inspiration time is not preset, patient's own breathing rhythm controls the inspiration time.

When the patient inhales from the mouthpiece, the pressure sensor responds and starts inhalation by providing a positive overpressure or opening of an inspiration valve. The nebulizer or aerosol system is supplied with compressed air overpressure of up to 40 mbar and the corticosteroid is aerosolized and discharged as a corticosteroid containing aerosol at a preselected flow rate and with preselected overpressure. The overpressure lasts for the entire inspiration time. When the inspiration time is preselected, the overpressure is automatically stopped or shut off because the compressed air supply is interrupted at the end of the inspiration time. After a period allocated for exhaling, the process is repeated on and off for the entire period of inhalation, preferably for less than 6 minutes. During the inhalation time, the whole dose is preferably aerosolized with some residue remaining in the nebulizer. The nebulizer can be a liquid or dry powder aerosol system.

Electronic equipment that may be attached to the nebulizer permits recordation of the inhalation process, storing of the records regarding the dose, time, air flow and overpressure and optimization of the treatment.

When this method of delivery is selected, during the inspiration time the aerosolized corticosteroid is forced under the overpressure into the lower lungs. When the overpressure is withdrawn and the patient exhales, the drug forced into the lower lungs is not easily displaced and remains there resulting in substantially higher deposition of the drug in the peripheral lungs than would happen with a normal breathing without overpressure. During the exhalation time, the small amount of the drug that is exhaled is the one that was in the upper lungs at the last moment of the inspiration time. Some fraction of this small amount may be deposited in the upper lungs or oropharyngeal area but most of the drug is exhaled to the outside of the mouth.

When the above treatment was performed on more then one hundred patients with inhalable fluticasone (2000 μg), administered once a day for 22 days, as described in Example 1, such treatment resulted in significant improvement of FEV1 by approximately 17% with simultaneous reduction in oral corticosteroid use by approximately 33%. Additionally, pulmonary inflammation, measured by exhaled nitric oxide, was reduced by approximately 44.5%. The details of the clinical study are described in Example 1.

### 1. Preferred Embodiments

In one preferred embodiment, a method for treatment of a patient suffering severe and uncontrollable asthma and requiring a concurrent treatment with oral corticosteroids comprises administering to an asthmatic patient an inhalable treatment comprising administration of an inhalable corticosteroid selected from the group consisting of fluticasone, beclomethasone dipropionate, budenoside, mometasone furoate, ciclesonide, flunisolide and triamcinolone acetonide and delivered as an aerosol containing the selected corticosteroid in amount from about 400 to about 4000 micrograms, where the aerosol is generated by a nebulizer device able to administer an aerosolized corticosteroid into lower lungs with a slight overpressure of maximum of 40 mbar or less, wherein such overpressure forces the aerosol into the lower lungs and results in deposition of more than 200 micrograms deposition of the corticosteroid into the lower lungs. This treatment further results in reduction of a need for concurrent treatment with oral steroid by at least 30%, in improvement of pulmonary functions and in reduction or elimination of oropharyngeal side effects.

In another embodiment, a selected corticosteroid is fluticasone administered in amount of about 4000 micrograms resulting in deposition of more then 200 micrograms in the lower lungs and preferably in the lung deposition larger than 400 micrograms.

In another embodiment, the requirement for concurrent treatment with oral steroids is completely eliminated in about 2 to 5 weeks and such treatment results in improvement of asthma symptoms, in increase of the FEV1 evidencing an improvement of pulmonary functions and in reduction of lung inflammation.

Another embodiment involves use of an inhalation system for control of breathing pattern, known as AKITA inhalation system and device.

In another embodiment, the method provides for inhalation treatment administered once, twice or three times a day, preferably only once a day with all benefits for asthma improvement.

In another embodiment, the method shortens time for delivery and the inhalation treatment is accomplished in less than 6 and maximum up to 10 minutes.

In another embodiment, the aerosol is provided that has a particle sizes primarily within a range of alveoli, bronchiole or bronchi with aerosol having particle sizes from about 2 to about 6 microns MMAD, preferably particle size from about 3 to about 5 microns MMAD.

C. Breath Actuated Treatment Protocol

When the second method for treatment is selected, a nebulizing system that is actuated by patient's breathing and the breath actuated nebulizer is used.

This nebulizer permits depositing aerosolized particles to specific areas of the lung by regulating aerosolization parameters of the device and by instituting a three prong inspiration time delivery.

Using this nebulizer system, the selected corticosteroid in the predetermined amount and volume is placed into the drug cartridge connected with a nebulizing device that also includes the mouthpiece and a spirometer. The predefined volume of aerosolized particles is delivered into the flow path through which the patient is inhaling. Inhalation time is preset to comprise a three predefined periods. The first predefined time period is for delivery of aerosol particle free air into the lungs at a flow rate that is also preset. The second predefined period is for delivery of a predefined volume of aerosolized particles of the corticosteroid, also at a preset flow rate. The third predefined period is for delivery of the second predefined time period of particle free air. Optionally, the first time period can also be set to zero seconds, meaning that the aerosolization will start immediately. During the inhalation, patient is instructed to begin inhalation and during each inspiration time, the three (or two) predetermined periods are repeated. At the end of the second particle free period, that is after the third predefined period, a patient is instructed to stop inhaling and exhale. The reason for the second predefined time period of aerosol particle free air delivery into the lungs at a flow rate within the preset flow rate range is to move the aerosolized particles out of the upper airway region. In that way the upper airway region (mouth, throat, oropharynx, larynx and trachea) is emptied from remaining aerosol particles and the deposition of the drug in this region is reduced.

The method also comprises a step of detecting when the subject is inhaling through the flow path and may further comprise steps of measuring and adapting the first, the second and the third predefined time period and/or the predefined volume of aerosolized particles to patient's health parameters

The method determines optimal time intervals for administration of the first particle-free air, for administration of an aerosolized inhalable corticosteroid and for administration of the second particle free air, wherein the cumulative time for these three time intervals correspond to one inspiration time. The time for each of the interval corresponds to from about 1 msec to about 10 sec, preferably from about 200 msec to about 5 seconds and may be the same or different for each interval.

The flow rate is a predetermined fixed flow rate, wherein the first predefined particle free air volume is up to about 0.15 liters, the predefined volume of aerosolized particles is up to about 3 liters and the second predefined particle free air volume is up to about 0.5 liters.

The nebulizer used for this method is equipped to detect when the subject is inhaling through the flow path and prevent flow through the flow path after providing the second predefined time period of aerosol particle free air.

### 1. Preferred Embodiments

In one embodiments, the invention concerns a method for treatment of a patient having severe and uncontrollable asthma requiring a concurrent treatment with oral corticosteroids and comprises a treatment protocol where a patient is treated with an aerosol comprising an inhalable corticosteroid selected from the group consisting of fluticasone, beclomethasone dipropionate, budenoside, mometasone furoate, ciclesonide, flunisolide and triamcinolone acetonide in amount from about 400 to about 4000 micrograms. The treatment protocol is set on one inspiration time divided into three predefined periods when the aerosol is administered in the second period. The three periods last from about 1 millisecond to about 10 seconds, preferably the first period lasts from 1 millisecond to about 1 second, the second period lasts from about 0.1 to about 10 seconds and the third period lasts from about 0.1 to about 5 seconds. An aerosolized particle free air is administered at a preset flow rate and a preset volume during the first periods followed by a second period when an aerosolized corticosteroid is administered at a preset flow rate and a preset volume and is followed by a third period when, again, an aerosolized particle free air is administered at a preset flow rate and a preset volume to clean the extrathoracic and tracheal airways of the corticosteroid and to force it deeper into the lungs. After the third period, the patient is instructed to stop inhaling and exhale. This protocol is repeated for about 6 to about 10 minutes or less, typically until a sufficient amount of the corticosteroid, that is larger than 200 micrograms, is nebulized and delivered into the lower lungs.

In another embodiment, the preset flow rate is an inspirational flow rate and is equal to or lower than 20 liters/minute.

In another embodiment, the preset flow rate is from about 3 to about 6 liters per minute and the aerosolized particle free air administered in the first period is administered at a preset volume of less then 150 ml in about a half a second time, the corticosteroid aerosol administered in the second period is administered at a volume of from about 200 to about 3000 ml or in a preset time of from 1 to about 10 seconds and the aerosolized particle free air administered in the third period is administered at a preset volume from about 200 to about 500 ml in about 0.3 to about 3.5 seconds time.

In another embodiment, the method results in reduction of oral corticosteroids requirement by at least 30% or in complete elimination for steroid drug and still resulting in improvement of asthma symptoms, in improvement of pulmonary functions determined by the FEV1 increase, in reduction of a lung inflammation and in overall reduction of oropharyngeal side effects.

In another embodiment, the selected corticosteroid is fluticasone administered in amount of about 200 micrograms once a day resulting in the lower lung deposition of fluticasone larger than 400 micrograms.

In another embodiment, the aerosol administered during the inspiration time and comprising three predefined periods is generated by breath actuated nebulizer.

In another embodiment the treatment protocol as outlined above is repeated once, twice or three times a day, the treatment is accomplished in less than 6 to 10 minutes.

In another embodiment, the aerosol has predetermined size of particles from about 2 to about 6 microns MMAD and preferably from about 3 to about 5 microns MMAD.

IV. Devices

Devices suitable for practicing the current invention have to have certain properties that meet the criteria for delivery of inhalable corticosteroids according to the invention.

A. Inhalation System for Control of Breathing Pattern

One inhalation system that is suitable for practicing the current invention is an inhalation system that comprises a compressor-driven jet nebulizer that controls the patient's breathing pattern during the inspiration phase. This system is highly effective for inhalation therapy requiring deposition of the aerosol into the lower lungs. During the inhalation, the system controls number of breaths, flow rate and inspirational volume. This ability to control these three parameters assure that the patient is given a correct dose.

The system further comprises an electronic means for individual personalization of a treatment protocol. The treatment protocol includes such parameters as individual's lung function measurements, optimum breathing pattern, desired drug dose to maintain or restore patients vital capacity (VC), expiratory resting volume (ERV) and forced expiratory volume per one second (FEV1). These parameters are individualized and stored on individual electronic record, sometime called Smart Card. These electronic records not only store the information for a treatment protocol and transfer this information to the system during treatment but also record and store the information for each of the treatments and show a possible error.

The Smart Card system may hold more than one treatment configuration and is fully encrypted. The Smart Card system is disclosed in the co-pending US patent application 2001/0037806 A1, published on Nov. 8, 2001, herein incorporated by reference in its entirety. The same or similar nebulizing system is disclosed in the U.S. Pat. No. 6,606,989, herein incorporated by reference in its entirety and is commercially available from Activaero GmbH, Gemünden (Wohra), Germany, under the trade name AKITA Inhalation System.

A similar but modified inhalation system further comprises, as a core element, a circular perforated membrane, that may be set to vibrate by a piezoelectric actuator. The vibrating motion of the membrane generates an alternating pressure that forces the nebulizing solution through a microarray of perforation in the membrane thus creating a fine aerosol having defined particle sizes. This system is similarly equipped with electronic means comprising the Smart Card, as described above. This system is commercially available from Activaero GmbH, Germünden (Wohra), Germany, under the trade name AKITA² APIXNEB Inhalation System.

Another modification of the inhalation system that can be used for practicing the current invention is the nebulizer that is triggered by the negative trigger pressure detected by a pressure sensor. This nebulizer comprises a compressor that provides a constant inhalation flow rate of 12 liters/minute during inspiration. This system has controlled flow, volume and nebulization timing. The Smart Card settings include inhalation volume, inhalation time per breath, nebulization time per one breath. This system is commercially available from Activaero GmbH, Gemünden (Wohra), Germany, under the trade name AKITA JET Inhalation System.

Other inhalation devices and systems that may be conveniently used or modified for use by the current invention are disclosed in the U.S. Pat. Nos. 6,401,710 E1, 6,463,929 B1, 6,571,791 B2,6,681,762 E1 and 7,077,125 B2 or in published applications 2006/0201499 A1 and 2007/0006883 A1, all herein incorporated by reference in their entirety.

B. Breath Actuated Nebulizer

A second nebulizing system that is suitable for practicing the current invention is a breath actuated nebulizer. This nebulizer is characterized by a passive flow and active volume control. Typically, it comprises a single use aerosol generator and a multi-use control device.

The device consists of an inhaler that is connected with a control unit. Inhaler itself is connected with nebulizer where the inhalable corticosteroid is nebulized into predetermined particles having sizes predominantly in the range from about 2 to about 6 μm, preferably between 3 and 5 μm using an aerosol generator. The filling volume of the nebulizer is approximately 4 ml. The aerosol generator is activated by pressure detection and is only activated during inspiration phase when the patient is inhaling the aerosolized corticosteroid. The pressure detection is controlled electronically.

This device is further equipped with means to permit administration of particle-free air, to permit the administration of an aerosolized inhalable corticosteroid, and to permit the second administration of the particle free air, each for a preselected time and volume, wherein the cumulative time for these three time intervals correspond to one inspiration time. The time for each of the interval corresponds to from about 1 msec to about 10 sec, preferably from about 200 msec to about 5 seconds.

The inhaler has an integrated flow and volume limited to about 15 liters/minute flow at a pressure of about 10 mbar or lower. When the underpressure at the mouthpiece is below 5 mbar, the flow rate is limited by a mechanical valve. The mechanical valve regulates the flow rate by a adjusting the cross section area. The unit is preset to a volume per one breath. One breath is set to be a time when one inspiration and one expiration occurs. After each inspiration time, the inspiration flow is blocked and expiration allowed. The inspiration flow is restored again for the next inspiration time during the next breath.

This device has various electronic components that permits its preprogramming and individualization meeting requirements of the individual asthmatic patients.

Exemplary device is disclosed in the pending patent application Ser. No. 12/183,747, filed on Jul. 31, 2008, herein incorporated by reference in its entirety. The device may be advantageously modified to provide a wide variety of variable conditions that may be easily individualized for patient's need. The modified device and method for its use is disclosed in the U.S. application Ser. No. 12/204,037, herein incorporated by reference in its entirety.

V. Clinical Protocol

The clinical protocol is provided for a double-blind, randomized, placebo-controlled phase II clinical trial to evaluate tolerability, safety and efficacy of inhaled fluticasone suspension using inhalation system for control of breathing pattern and AKITA protocol (AICS) in subjects with asthma requiring chronic oral corticosteroid treatment.

Objectives of the clinical study is to evaluate the tolerability and safety of high dose nebulized fluticasone delivered with AKITA device and to study the efficacy of high dose nebulized fluticasone in reducing the need for oral corticosteroid (OCS) therapy.

Asthma subjects are maintained on a chronic dose of oral corticosteroids. The total study period is 24 weeks.

The study schedule includes 2-weeks screening period, 2-weeks treatment and tolerability period, 18-weeks treatment period, including 14-weeks OCS taper period with 7 downward tapering steps, and a follow-up visit 2 weeks after the last dose of study drug. All subjects are provided with oral prednisone to use for the duration of study participation.

One hundred or one hundred and twenty subjects (50 or 60 per treatment group at approximately 25 asthma centers in three countries are assigned randomly to treatment with either high dose nebulized fluticasone or placebo administered BID.

4 mg fluticasone suspension (2 mL of 2 mg/mL fluticasone suspensions or matching placebo (2 mL of 0.9% sterile, normal saline in suspension) is delivered via the Activaero Akita Jet nebulizer twice daily.

Study requires that the subjects for this study are and must be older than 12 years and younger than 75 years of age and exhibit FEV1 from 30% to 90% of predicted (pre-albuterol treatment) who have been treated with inhaled and oral corticosteroids for more than 6 months. The average daily dose of OCS must be within 5 to 70 mg of prednisone equivalent.

Study endpoints evaluation includes reduction of OCS (oral corticosteroid, e.g. prednisone), improvement of asthma control, pulmonary function (FEV1), and reduction of pulmonary inflammation determined by amount of exhaled nitric oxide.

This therapy, for the first time, provides asthma control in the severe and uncontrollable asthma population without the use of systemic, oral steroids. The clinical side effects of systemic corticosteroids, such as bone loss, diabetes, cataract, obesity, etc., are avoided.

The therapy delivers high amounts of the corticosteroid to the lung, without depositing substantial amounts to the oropharynx.

### EXAMPLE 1

### Fluticasone Study

This example describes fluticasone lung delivery therapy using AKITA protocol to reduce dependence on oral corticosteroids, along with improvement of pulmonary function and reduction of lung inflammation

The study was performed at the Davos Pulmonary Clinic situated in Switzerland that treats hundreds of severe asthma patients for a planned duration of 2 to 8 weeks each year. The patients are typically chronically maintained on oral and inhaled asthma medication, and are not in an exacerbation. Patients are treated with an average prednisone (OCS) dose of 20-25 mg/day with substantial variability).

More than one hundred patients suffering from severe asthma that are chronically maintained on OCS (oral corticosteroids) were being treated with an AKITA protocol, consisting of a once daily inhalation of high dose fluticasone (2000 μg fluticasone filled dose, administered via AKITA nebulizer).

Patients were selected on the basis of their need for OCS, and on the basis of poor symptom control. All those patients have previously been treated with both inhaled corticosteroid/beta agonist combinations using meter dose inhalers and dry powder inhalers and oral steroids, at an average of 22.9 mg/day prednisolone or equivalents. None of the patient had adequate control of asthma symptoms and adequate pulmonary function, as determined by FEV1.

Treatment with AICS was conducted for 2 to 8 weeks, with 3 weeks on average, under in-house conditions, as a once daily inhalation of fluticasone 2000 μg, in an open-label, uncontrolled fashion. Of the nominal, filled dose of 2000 μg, an approximate 500-700 μg reached the central lungs, with a similar amount remaining in the nebulizer. The remaining, small amount remained in the oropharynx or was exhaled.

Treatment duration for the 112 patients analyzed for 2007 was 7 to 53 days. Following outcome parameters were determined: Pulmonary Function (FEV1), Exhaled Nitric Oxide (PeNO), OCS dose, and asthma control.

On average, the daily treatment was applied for 22 days (range 7 to 53 days), while patients were hospitalized and monitored.

Outcome of the study was unexpected and surprising. Asthma control was achieved more rapidly and consistently with AICS treatment, while oral corticosteroids were reduced. Specifically, both the FEV1 and reduction of oral steroids showed highly significant changes versus baseline, p<0.0001). OCS was reduced from a mean of 22.9 mg/day to 15.6 mg/day. The average FEV1 improved by 17.2% (or 340 ml). The oral steroids were reduced by 7.6 mg (33.2% reduction) on average. In addition, the pulmonary inflammation, as measured by exhaled nitric oxide, was reduced by 44.5% (p<0.0001). The use and handling of the AKITA nebulizing device for daily inhalation was well accepted by the patients and contributed to protocol compliance.

What is claimed is:

1. A method for treatment of a patient having severe and uncontrollable asthma requiring a concurrent treatment with oral corticosteroids, said method comprising administering to the patient in need thereof
an inhalable treatment comprising administration of an inhalable corticosteroid selected from the group consisting of fluticasone, beclomethasone dipropionate, budenoside, mometasone furoate, ciclesonide, flunisolide and triamcinolone acetonide as an aerosol comprising said corticosteroid in amount from about 400 to about 4000 micrograms,
using a nebulizer device able to administer an aerosolized corticosteroid into lower lungs with overpressure of 40 mbar or less,
wherein said overpressure results in a larger than 200 micrograms deposition of said corticosteroid into the lower lungs, and
wherein said inhalable treatment results in reduction of at least 30% of oral steroids requirement and reduction of oropharyngeal side effects.

2. The method of claim 1 wherein said corticosteroid is fluticasone.

3. The method of claim 2 wherein the lung deposition of fluticasone or another steroid is larger than 400 micrograms.

4. The method of claim 1 wherein the requirement for concurrent treatment with oral steroids is eliminated.

5. The method of claim 1 wherein said inhalable treatment with inhalable corticosteroids results in improvement of asthma symptoms, in improvement of pulmonary functions determined by an increase in a forced expiratory volume (FEV1) and in reduction of a lung inflammation.

6. The method of claim 1 wherein said device is an inhalation system for control of breathing pattern (AKITA)

7. The method of claim 1 wherein said inhalation treatment is administered once, twice or three times a day.

8. The method of claim 7 wherein said inhalation treatment is accomplished in less than 10 minutes.

9. The method of claim 8 wherein said inhalation treatment is accomplished in less than 6 minutes.

10. The method of claim 1 wherein said aerosol has particle sizes from about 2 to about 6 microns MMAD.

11. The method of claim 10 wherein said particle size is from about 3 to about 5 microns MMAD.

12. A method for treatment of a patient having severe and uncontrollable asthma requiring a concurrent treatment with oral corticosteroids, said method comprising a treatment protocol comprising administering to a patient in need thereof
an inhalable treatment comprising administration of an inhalable corticosteroid selected from the group consisting of fluticasone, beclomethasone dipropionate, budenoside, mometasone furoate, ciclesonide, flunisolide and triamcinolone acetonide as an aerosol comprising said corticosteroid in amount from about 400 to about 4000 micrograms,
wherein said aerosol is administered during an inspiration time comprising three predefined periods
wherein in the first period lasting from about 1 millisecond to about 1 second, an aerosolized particle free air is administered at a preset flow rate and at a preset volume;
wherein in the second period lasting from about 0.1 to about 5 seconds, an aerosolized corticosteroid is administered at a preset flow rate and at a preset volume;
wherein in the third period, lasting from about 1 millisecond to about 10 seconds, an aerosolized particle free air is administered at a preset flow rate and at a preset volume;
wherein after the third period, the patient is instructed to stop inhaling and exhale;
wherein said protocol is repeated for about 6 to about 10 minutes or less; and
wherein said treatment results in a larger than 200 micrograms deposition of said corticosteroid into the lower lungs.

13. The method of claim 12 wherein said preset flow rate is an inspirational flow rate and is equal or below 20 liters/min.

14. The method of claim 13 wherein said aerosolized particle free air administered in the first period is administered at a preset volume of less then 150 ml in about 0.5 second time.

15. The method of claim 13 wherein said corticosteroid aerosol administered in the second period is administered at a volume of from about 200 to about 3000 ml or in a preset time of from 1 to about 10 seconds.

16. The method of claim 13 wherein said aerosolized particle free air administered in the third period is administered at a preset volume from about 200 to about 500 ml in about 0.3 to about 3.5 seconds time.

17. The method of claim 12 resulting in reduction of oral corticosteroids requirement by at least 30% and in reduction of oropharyngeal side effects.

18. The method of claim 17 wherein said corticosteroid is fluticasone administered in amount of about 200 micrograms once a day.

19. The method of claim 18 wherein the lung deposition of fluticasone is larger than 400 micrograms.

20. The method of claim 19 wherein the requirement for concurrent treatment with oral steroids is eliminated.

21. The method of claim 12 wherein said treatment protocol results in improvement of asthma symptoms, in improvement of pulmonary functions determined by increase in the FEV1 and in reduction of lung inflammation.

22. The method of claim 12 wherein said wherein said aerosol administered during the inspiration time and comprising three predefined periods is generated by a breath actuated nebulizer.

23. The method of claim 12 wherein said treatment protocol is repeated once, twice or three times a day.

24. The method of claim 12 wherein said treatment protocol is accomplished in less than 10 minutes.

25. The method of claim 24 wherein said treatment protocol is accomplished in less than 6 minutes.

26. The method of claim 12 wherein said corticosteroid aerosol has particle sizes from about 2 to about 6 microns MMAD.

27. The method of claim 26 wherein said aerosol has particle sizes from about 3 to about 5 microns MMAD.
